(19) United States
(12) Patent Application Publication
(10) Pub. No.: US 2026/0331305 A1
(43) Pub. Date: Oct. 22, 2026

(54) SYSTEM FOR DEVELOPING AND DISTRIBUTING NUTRIENT-COMPOUNDED PHARMACEUTICALS

(71) Applicant: United States Performance Center, LLC, Charlotte, NC (US)

(72) Inventors: David Koerner, Waxhaw, NC (US); Irwin Belk, II, Charlotte, NC (US)

(73) Assignee: United States Performance Center, LLC, Charlotte, NC (US)

(21) Appl. No.: 19/655,233

(22) Filed: Apr. 22, 2026

Related U.S. Application Data
(60) Provisional application No. 63/792,401, filed on Apr. 22, 2025.

(51) Int. Cl. G16H 70/40; A61J 1/03; G16C 20/20; G16C 20/90

(52) U.S. Cl. CPC G16H 70/40; A61J 1/035; G16C 20/20; G16C 20/90

(57) ABSTRACT

A computer system and method for developing and providing a drug-nutrient combination are provided, comprising: a computerized system having a processor and a memory storing instructions that, when executed by the processor, cause the system to: retrieve pharmaceutical research from one or more databases, the pharmaceutical research including information regarding a pharmaceutical drug, drug mechanisms of action, potential side effects, and dosing; retrieve medical research directed to an impact of combining the pharmaceutical drug with a nutrient; analyze the pharmaceutical research and the medical research to identify a nutrient having a synergistic impact on the pharmaceutical drug; determine a drug-nutrient combination based on the pharmaceutical research and the medical research, the drug-nutrient combination including the pharmaceutical drug and the nutrient; and generate a recommendation for the drug-nutrient combination.

### RELATED APPLICATIONS

This application claims priority to U.S. Provisional Application No. 63/792,401, filed Apr. 22, 2025, the entire contents of which are incorporated herein by reference.

### BACKGROUND OF THE INVENTION

### 1. Field of Invention

This is a method, process and system directed to a novel technique for developing and distributing nutrient-compounded pharmaceuticals that combine pharmaceutical drugs with dietary nutrients for enhanced efficacy and reduced side effects.

### 2. Description of the Related Art

Approximately two-thirds of the United States population use prescription medications and will spend nearly half of their lives taking medications. While medications are intended to improve health and wellness, their effectiveness can vary significantly between individuals due to factors such as age, body size, genetics, metabolism, existing health conditions, stress, inflammation, and diet. Additionally, all medications carry a risk of side effects or adverse reactions, which can lead to significant healthcare burdens. Further, individual responses to medication can differ substantially based on genetic variations that affect drug metabolism, receptor sensitivity, and enzyme activity, making standardized dosing approaches suboptimal for many patients. Enhanced drug effectiveness through nutrient combinations may allow for smaller doses of medication to be prescribed, thereby reducing associated side effects and lowering medication costs.

Dietary nutrients may offer substantial benefits for reducing the adverse effects associated with prescription medications. The term "Nutritional Pharmacology" refers to the use of high concentrations of dietary nutrients for therapeutic benefit. Nutritional deficiencies can impact how a medication is absorbed, metabolized, and excreted, and this directly impacts the effectiveness of the medication. Conversely, medications can negatively impact the absorption and metabolism of specific nutrients and directly cause deficiencies. In order to improve the effectiveness of a prescribed medication with known adverse side effects, the medication can be strategically packaged with the appropriate dose of nutrients that will counteract the negative effects and enhance the positive effects.

Several classes of nutrients have been shown to improve the effectiveness of cancer treatments and have also been shown to reduce the side effects associated with chemotherapy treatments. For example, Omega-3 fatty acids have shown considerable benefit when supplemented with existing cancer treatments and could therefore be packaged with the existing treatments in the appropriate clinical doses to improve patient treatment responses. Additionally, researchers have found that opium users have a disruption of vitamin metabolism, suggesting that combining vitamin compounds with existing pharmaceuticals may lead to more successful outcomes when treating opioid addiction. Furthermore, folate has been shown to increase the effectiveness of antidepressant medications.

The study of nutrient-drug interactions is a growing field that explores how pharmaceutical drugs can influence nutrient status within the body, and conversely, how an individual's nutritional status may impact the effects of a drug. Evidence suggests that numerous drugs may cause deficiencies in vitamins, minerals, fatty acids, and amino acids, potentially leading to further undesirable effects within the body. Dietary nutrients have shown potential in reducing adverse effects associated with prescription medications. Nutritional deficiencies can impact how a medication is absorbed, metabolized, and excreted, directly affecting its effectiveness. Conversely, medications can negatively impact the absorption and metabolism of specific nutrients, causing deficiencies.

Current medical practices typically prescribe standard pharmaceutical drugs without consideration of beneficial nutrient combinations that may enhance drug effectiveness or reduce side effects. Even when practitioners are aware of beneficial nutrient combinations, they are often suggested to patients rather than prescribed as part of a unified treatment approach. This practice may be improved through the development of nutrient-compounded pharmaceutical formulations that strategically combine medications with appropriate doses of nutrients.

A system is needed that provides for the development and distribution of nutrient-compounded pharmaceuticals that address the challenges associated with drug-induced nutrient depletion and suboptimal treatment outcomes. Certain pharmaceutical drugs reliably deplete specific nutrients in a large proportion of patients taking those medications, and the addition of the depleted nutrient to the pharmaceutical regimen can reduce side effects for a significant population of patients. For example, statins are known to deplete coenzyme Q10in many patients, and proton pump inhibitors commonly reduce magnesium and vitamin B12 levels. A system is needed that provides for fixed combinations of pharmaceutical drugs and nutrients that reduce pill burden and improve patient adherence to treatment regimens. A system is needed that provides for predictable synergy between pharmaceutical drugs and nutrient compounds. A system is needed that provides for non-specialized combinations that scale easily for widespread manufacturing and distribution.

It is an object of the present system to provide for improvements in pharmaceutical delivery by combining traditional drugs with specific dietary nutrients to enhance drug effectiveness and reduce side effects.

It is another object of the present system to provide a system that reduces pill burden and improves patient adherence. Reduced side effects from nutrient-compounded pharmaceuticals may lead to improved patient compliance with treatment regimens. In some cases, improved compliance may result in fewer doctor visits and cost savings for patients and insurance providers.

It is an object of the present system to provide for the use of medical research and analysis to identify optimal nutrient-drug combinations based on factors including genetic makeup, nutritional status, and gut microbiome composition.

It is an object of the present system to provide for nutrient-compounded pharmaceuticals that may be manufactured on a large scale based on population trend data for combinations that scale easily for widespread manufacturing and distribution.

It is an object of the present system to provide for predictable synergy between pharmaceutical drugs and nutrient compounds.

### BRIEF SUMMARY OF THE INVENTION

The above objectives are accomplished by providing a method, process and computer system for developing and providing a drug-nutrient combination. The computer system includes a computerized system having a processor and a memory storing instructions that, when executed by the processor, cause the system to retrieve pharmaceutical research from one or more databases, the pharmaceutical research including information regarding a pharmaceutical drug, drug mechanisms of action, potential side effects, and dosing. The system retrieves medical research directed to an impact of combining the pharmaceutical drug with a nutrient. The system analyzes the pharmaceutical research and the medical research to identify a nutrient having a synergistic impact on the pharmaceutical drug. The system determines a drug-nutrient combination based on the pharmaceutical research and the medical research, the drug-nutrient combination including the pharmaceutical drug and the nutrient. The system generates a recommendation for the drug-nutrient combination. The methos and system includes the design, creation, manufacturing and distributing of the compound or joint compounds.

According to other aspects of the present disclosure, the computer system may include retrieving a nutrient analysis data including information regarding nutritional deficiencies associated with the pharmaceutical drug. The computer system may be adapted to retrieve patient analysis data including disease-related patient research to identify appropriate nutrients for a patient population. The patient analysis data may include microbiome analysis data derived from DNA sequencing methods. The nutrient may be selected from the group consisting of: vitamins, minerals, amino acids, omega-3 fatty acids, prebiotics, probiotics, antioxidants and others. The pharmaceutical drug may be selected from the group consisting of: chemotherapy drugs, opioid addiction treatment drugs, antidepressants, HIV drugs, cardiovascular disease drugs, diabetes drugs and others. The computer system may be adapted to generate a formulation specification for compounding the pharmaceutical drug with the nutrient into a single dosage form. The computer system may be adapted to generate a packaging specification for co-packaging the pharmaceutical drug and the nutrient in separate compartments of a package.

According to another aspect of the present disclosure, a computer system for identifying a compound is provided. The computer system includes a processor and a set of computer readable instructions that, when executed by the processor, cause the system to access a pharmaceutical and nutrient database including listings of pharmaceutical drugs with applications, doses, interactions, and side effects, and listings of nutrients with applications, doses, and synergistic effects with pharmaceutical drugs. The system receives an indication of a pharmaceutical drug. The system retrieves, from the pharmaceutical and nutrient database, information regarding the pharmaceutical drug and one or more nutrients. The system evaluates compound properties including an interaction between the pharmaceutical drug and a nutrient. The system determines suitability of a compound including the pharmaceutical drug and a selected nutrient based on the compound properties. The system outputs a compound formulation including the pharmaceutical drug and the selected nutrient.

According to other aspects of the present disclosure, the compound formulation may include a dose of the pharmaceutical drug and an amount of the selected nutrient configured to provide a synergistic effect. The computer system may be adapted to retrieve medical research including clinical findings relevant to combining the pharmaceutical drug with the one or more nutrients. The compound formulation may specify incorporating the selected nutrient into a matrix of a tablet or capsule containing the pharmaceutical drug.

According to another aspect of the present disclosure, a computerized system for managing a drug-nutrient combination is provided. The computerized system includes a computer system having a processor and a set of computer readable instructions that, when executed by the processor, cause the system to receive information from a pharmaceutical and nutrient database and medical research. The system processes and analyzes the information to determine a formulation or combination of a nutrient and a pharmaceutical drug. The system determines whether to compound the pharmaceutical drug with the nutrient into a single dosage form or to combine the nutrient with the pharmaceutical drug in packaging. The system generates a formulation for delivery to a pharmacy, the formulation including the pharmaceutical drug combined with the nutrient, based on a determination to compound. The system generates a packaging configuration including the pharmaceutical drug and the nutrient in separate compartments, based on a determination to combine in packaging.

According to other aspects of the present disclosure, the packaging configuration may include a blister pack having a packet section divided into multiple compartments, each compartment containing a nutraceutical and a pharmaceutical drug. The blister pack may include at least one perforation to allow separation of individual doses. The computer system may be adapted to utilize machine learning algorithms to analyze research data and identify patterns between nutrient compounds and pharmaceutical efficacy. The computer system may be adapted to utilize natural language processing techniques to extract information from research documents and integrate the information into a decision-making process for determining the formulation or combination.

According to another aspect of the present disclosure, a method for developing and providing a drug-nutrient combination is provided. The method includes retrieving, by a processor, pharmaceutical research from one or more databases, the pharmaceutical research including information regarding a pharmaceutical drug, drug mechanisms of action, potential side effects, and dosing. The method includes retrieving, by the processor, medical research directed to an impact of combining the pharmaceutical drug with a nutrient. The method includes analyzing, by the processor, the pharmaceutical research and the medical research to identify a nutrient having a synergistic impact on the pharmaceutical drug. The method includes determining, by the processor, a drug-nutrient combination based on the pharmaceutical research and the medical research, the drug-nutrient combination including the pharmaceutical drug and the nutrient. The method includes generating, by the processor, a recommendation for the drug-nutrient combination.

According to other aspects of the present disclosure, the method may further include retrieving nutrient analysis data including information regarding nutritional deficiencies associated with the pharmaceutical drug, and selecting the nutrient based on the nutrient analysis data to address the nutritional deficiencies. The method may further include generating a formulation specification for compounding the pharmaceutical drug with the nutrient into a single dosage form, or generating a packaging specification for co-packaging the pharmaceutical drug and the nutrient in separate compartments of a package.

### BRIEF DESCRIPTION OF THE SEVERAL VIEWS OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 is a flowchart of aspects of the system for developing nutrient-compounded pharmaceuticals;

FIG. 2 is a top view of a blister pack layout of aspects of the system;

FIG. 3 is a flowchart of aspects of the system for creating nutrient-compounded pharmaceuticals; and

FIG. 4 is a system diagram of aspects of the system for creating nutrient-compounded pharmaceuticals.

While each of the drawing figures depicts a particular embodiment for purposes of depicting a clear example, other embodiments may omit, add to, reorder, and/or modify any of the elements shown in the drawing figures. For purposes of depicting clear examples, one or more figures may be described with reference to one or more other figures, but using the particular arrangement depicted in the one or more other figures is not required in other embodiments. The drawings and schematic representations are intended to support the understanding of the invention. These may not be to scale and are not intended to limit the invention to any particular layout, connectivity, or architectural implementation. Correspondence between drawing elements and described components is provided for illustrative purposes and should not be interpreted to limit the claim scope.

### DETAILED DESCRIPTION OF THE INVENTION

In the following description, for the purposes of explanation, numerous specific details are set forth in order to provide a thorough understanding of the present disclosure. It will be apparent, however, that the present disclosure may be practiced without these specific details. In other instances, well-known structures and devices are shown in block diagram form in order to avoid unnecessarily obscuring the present disclosure. Modifiers such as “first” and “second” may be used to differentiate elements, but the modifiers do not necessarily indicate any particular order.

With reference to the drawings, the invention will now be described in more detail.

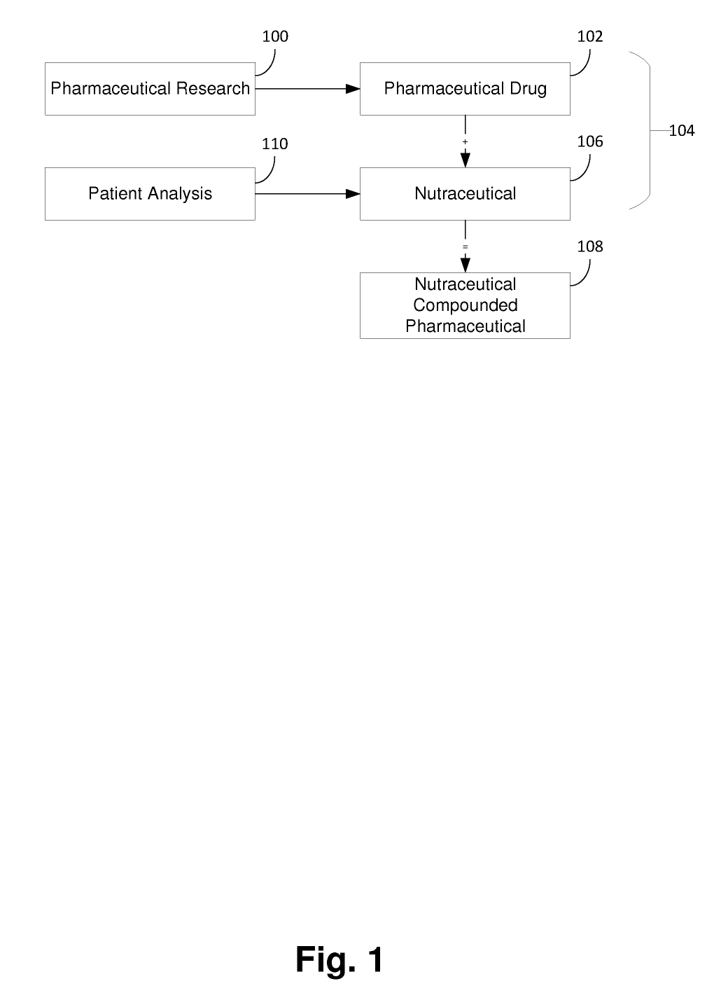

Referring to FIG. 1, a flowchart of a computerized system and method for creating nutrient combined or compounded pharmaceuticals is shown. The system begins with a step 100, where pharmaceutical research is retrieved from third party sources. Pharmaceutical research involves a systematic process of discovering, developing, and testing new drugs to treat various medical conditions. In one embodiment, the pharmaceutical research includes several stages, such as target identification, lead compound discovery, preclinical testing, and clinical trials. During target identification, researchers identify specific molecules or biological processes that play a role in a disease. This involves studying the underlying mechanisms of diseases and identifying points of intervention.

Once a target is identified, researchers screen large libraries of compounds to find those that interact with the target in a desired way. The pharmaceutical research at step 100 can be used to determine the nutraceutical and pharmaceutical combination / compound, the dose, the processing method, and other parameters. The pharmaceutical research can include compound discovery that involves optimizing promising compounds to improve efficacy, safety, and other properties. The pharmaceutical research can include preclinical testing conducted to assess the safety and efficacy of drug candidates in animal models before moving to human trials. The pharmaceutical research can include clinical trials conducted in multiple phases to evaluate the safety and efficacy of a drug in humans. The pharmaceutical research at step 100 provides information about drug mechanisms of action, potential side effects, contraindications, and dosing.

From step 100, the system proceeds to a step 102, where a pharmaceutical drug is provided based on the pharmaceutical research. The pharmaceutical drug at step 102 serves as the foundation for the nutrient compounded pharmaceutical. The pharmaceutical drug can be selected from chemotherapy drugs, opioid addiction treatment drugs, antidepressants, HIV drugs, CVD disease drugs, and diabetes drugs and more.

The system includes a step 104, which represents a nutrient analysis component that is part of the overall process. The nutrient analysis at step 104 involves a comprehensive assessment of nutritional status using various methods and techniques. In one embodiment, the step 104 includes using research that includes conducting a physical examination over a set of subjects to identify signs of nutritional deficiencies or excesses. In one embodiment, the step 104 includes research that assesses weight, height, body mass index (BMI), and body composition. These anthropometric measurements provide valuable insights into nutritional status and areas of concern. In one embodiment, the step 104 includes using research that includes performing blood tests and other laboratory assessments to measure levels of essential nutrients such as vitamins, minerals, and proteins. For example, a complete blood count (CBC) can be conducted to assess iron levels, while specific vitamin assays can be used to measure levels of vitamins D, B12, or folate. In one embodiment, the step 104 includes using research that determines the genetic makeup through genetic testing which reveals specific genetic variations that influence nutrient metabolism or absorption, allowing for a more effective approach to nutrient supplementation.

The method includes a step 106, where a nutraceutical is selected based on the pharmaceutical research and nutrient analysis. The nutraceutical at step 106 is determined to have synergistic impact on the pharmaceutical drug according to the research. In one embodiment, step 106 utilizes metabolomics and pharmacomicrobiomics techniques to support the selection or creation of a targeted nutrient combination or compound with a pharmaceutical. Metabolomics involves the study of small molecules (metabolites) present in biological samples, providing insights into metabolic state. Pharmacomicrobiomics focuses on how the gut microbiome influences drug metabolism and efficacy. By integrating these approaches, step 106 allows one version of the system to identify specific nutrients that can enhance drug effectiveness or mitigate potential side effects.

In one configuration, step 106 involves selecting specific amino acids for inclusion in the nutrient compound. In one configuration, step 106 results in the system considering the inclusion of prebiotics or probiotics based on the results of patient data microbiome analyses. In one configuration, step 106 involves identifying antioxidants for inclusion in the nutrient combination or compound. The nutrient combination or compound from step 106 is tailored not only to address nutritional needs but also to complement the pharmaceutical drug from step 102. For example, if the drug is known to deplete certain nutrients, step 106 adds higher doses of these nutrients to the compound or combination to prevent deficiencies.

The system further includes a step 108, where a nutraceutical compounded pharmaceutical is created by combining the pharmaceutical drug from step 102 with the nutraceutical from step 106. The nutraceutical compounded pharmaceutical at step 108 provides a final product that combines the pharmaceutical drug with specific dietary nutrients to enhance drug effectiveness and reduce side effects. In one embodiment, step 108 physically combines the pharmaceutical drug and nutrient compound into a single dosage form. For example, the nutrient compound can be incorporated into the matrix of a tablet or capsule containing the pharmaceutical drug. In other embodiments, step 108 involves creating a co-packaged product where the pharmaceutical drug and nutrient compound are provided separately but intended to be taken together.

In one embodiment, the system includes a step 110, which represents patient analysis that feeds into the nutraceutical selection at step 106. The patient analysis at step 110 includes information about mass disease-related patient research that can be used to identify and recommend appropriate nutrients. In one example, used for individualized medicine, the step 110 includes microbiome analysis involving sample collection from test subjects or existing medical data taken in the normal treatment of patients. This typically involves collecting stool samples, as stool samples provide a representative view of the gut microbiome. The samples are collected using specialized kits designed to preserve the microbial composition during transport to a laboratory for analysis.

In one example, the step 110 includes DNA sequencing methods such as 16S rRNA sequencing or metagenomic sequencing to characterize microbiomes. The 16S rRNA sequencing provides information about the taxonomic composition of the microbiome, identifying the types and relative abundances of different bacterial species present. Metagenomic sequencing offers a more comprehensive view, providing information not only about the microbial species present but also about functional capabilities.

In one example, the step 110 includes bioinformatics analysis of the sequencing data using specialized software and algorithms to process the raw sequencing data. The analysis compares microbiome profiles to reference databases of healthy microbiomes or those associated with specific health conditions.

In one example, the step 110 includes identifying dysbiosis or imbalances in the microbiome including detecting overgrowth of potentially harmful bacteria, absence of beneficial bacteria, or overall reduced diversity. The analysis also identifies specific microbial functions that are over- or under-represented in test subjects' microbiomes. The results of the microbiome analysis at step 110 contribute to the overall method used by the system. For example, if the analysis reveals a deficiency in bacteria that produce short-chain fatty acids, the method incorporates prebiotics or specific dietary fibers that support the growth of beneficial bacteria. The microbiome analysis provides insights into how the gut microbiome interacts with pharmaceutical drugs, as certain microbial species influence drug metabolism, affecting the bioavailability and efficacy of medications.

The flowchart shows that pharmaceutical research at step 100 flows to the pharmaceutical drug at step 102, which then connects to the nutraceutical at step 106. The patient analysis at step 110 also connects to the nutraceutical at step 106. The nutraceutical at step 106 then flows to the nutraceutical compounded pharmaceutical at step 108. A bracket labeled step 104 encompasses the pharmaceutical drug at step 102, the nutraceutical at step 106, and the nutraceutical compounded pharmaceutical at step 108, indicating these components are part of the nutrient analysis process.

In one embodiment, the system allows for iterative refinement of the nutrient compounded pharmaceutical based on feedback and results. The flow of the system demonstrates how each step contributes to the final product. The pharmaceutical research at step 100 provides the base drug, while the analyses at steps 104 and 110 inform the creation of a tailored nutrient compound at step 106. The combination of these elements at step 108 results in a nutrient compounded pharmaceutical that is optimized for patients.

In one embodiment, the system continuously retrieves research information from pharmaceutical databases, clinical trial repositories, peer-reviewed publications and other sources to update and refine the suggested nutrient-pharmaceutical compounds and combinations. The system can employs machine learning algorithms that analyze newly published research data, clinical outcomes, and patient feedback to identify emerging patterns and correlations between specific nutrient compounds and pharmaceutical efficacy. In one embodiment, the system allows attending medical professionals to input patient information, treatment outcomes, and clinical observations directly into the system to support the feedback and learning functionality.

As new research becomes available, the machine learning models are retrained to incorporate the latest findings, allowing the system to adapt its recommendations over time. In one configuration, the system utilizes natural language processing techniques to extract relevant information from unstructured research documents and integrate this information into the decision-making process. The continuous learning capability enables the system to identify previously unknown synergistic relationships between nutrients and pharmaceutical drugs, as well as to detect potential adverse interactions that may not have been apparent in earlier research. In one embodiment, the system maintains a feedback loop where treatment outcomes are collected and analyzed to validate or refine the nutrient compound recommendations. This iterative learning process allows the system to improve the accuracy and effectiveness of its suggestions as more data becomes available, resulting in increasingly optimized nutrient-compounded pharmaceuticals over time.

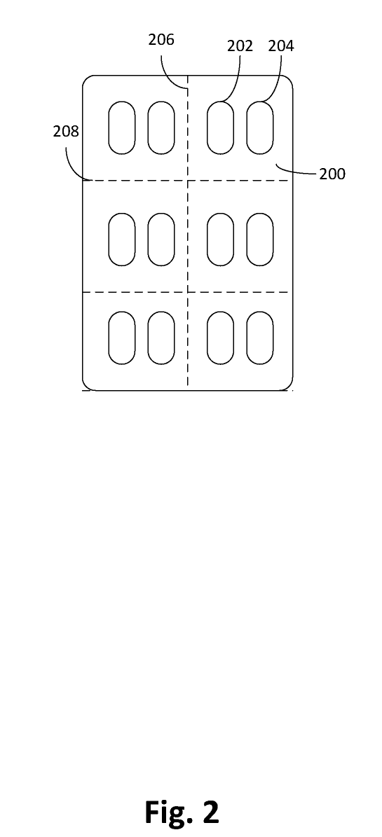

Referring to FIG. 2, a top view of one example of a pharmaceutical tablet blister pack layout for a nutrient combined with a pharmaceutical is shown. The layout includes a packet section 200, which is divided into multiple compartments arranged in a grid pattern. Each compartment can contain two distinct elements: a nutraceutical 202 and a pharmaceutical drug 204. The nutraceutical 202 and pharmaceutical drug 204 represent the nutrient compound and the pharmaceutical drug, respectively, that are combined to create the nutrient pharmaceutical combination. The nutraceutical 202 and pharmaceutical drug 204 can be positioned side by side within each compartment of the packet section 200.

In one embodiment, the nutraceutical 202 and pharmaceutical drug 204 are physically separated within each compartment, allowing for combination at the time of consumption. This separation helps maintain the stability and efficacy of both components until the components are ready to be used.

The blister pack layout can incorporate a perforation 206 extending horizontally across the packet section 200, which allows for easy separation of individual rows of doses. A perforation 208 extends vertically down the center of the packet section 200, enabling separation of the pack into left and right sections. The perforation 206 and perforation 208 facilitate convenient dispensing and administration of the nutrient compounded pharmaceuticals by allowing users to tear off individual doses or sections as needed.

In one embodiment, the combination is distributed in a single bag or a bag with sections for each element. The bag can include both elements in a pocket defined in the bag or can include two or more pockets. In one configuration, the combination is delivered in a calendar pack format, which is a form of a blister pack where each blister is labeled with the date of the month or day of the week.

In one embodiment, the combination is provided in a unit dose package that contains one discrete pharmaceutical dosage form, such as one tablet, one volume of liquid, one mass of ointment, etc. A unit dose package contains the particular dose of the drug. A single unit package is also a unit dose or single dose package if the single unit package contains the particular dose of the drug or combination of pharmaceuticals and nutraceuticals. A unit dose package can contain two or more tablets of a product.

In one configuration, the nutraceutical is compounded directly with the pharmaceutical and delivered in a single consumable. The single consumable can include tablets, liquids, capsules, or powders. Pharmaceuticals and nutraceuticals can be delivered in liquid form using ampules, bioprocess containers, and bottles. These liquid delivery forms can be single dose containers that are intended to be used by a single patient. The containers can come in one or several sizes, and depending on size, a single patient can be treated with one or several containers in order to receive the correct dose.

In powder form, sachets cen be designed to provide accurate and easy access to the powdered medication, making measurement and administration of the correct dosage straightforward. Sachet packaging can enhance patient compliance and safety by ensuring that individuals receive the right amount of medication as prescribed.

The packaging can include secondary packaging, which is the outer layer of packaging that surrounds the primary packaging. Secondary packaging protects the drug, provides information, and helps with handling and distribution. Secondary packaging can be made from plastics such as polyethylene (PE), polypropylene (PP), and polyethylene terephthalate (PET), low-density polyethylene (LDPE) laminates, aluminum foil, cardboard, and glass. Secondary packaging can take the form of cardboard cartons, cardboard boxes, cardboard or plastic crates, trays, paperboard cartons, shrink wrapped bundles, trayed PET bottles held with shrink wrap, or taped or glued cardboard cartons. Secondary packaging provides protection to the primary packaging during storage and transportation to reduce the risk of breakages of the primary packaging.

Blister package material can be chosen based on barrier properties. Common blister barrier materials include polyvinyl chloride, polyvinyl dichloride, polychlorotrifluoroethylene (Aclar), aluminum foil, and cyclic olefin copolymer. Aluminum foil offers low moisture penetrance, while polyvinyl chloride is often used as a blister material due to low cost and ease of production. In one embodiment, the packaging materials and construction do not absorb, adsorb, or otherwise deleteriously affect the contents of the packaging providing an environment for transportation and storage of the pharmaceuticals and nutrients.

Information can be available to practitioners indicating the stability and compatibility of contents with various packaging materials. Packages are constructed so that the packages do not deteriorate with normal handling. The packaging is easy to open and use, and use of the packaging requires little or no special training or experience. Unless the package contains a drug to be added to a parenteral fluid or otherwise used in compounding a finished dosage form, the package allows the contents to be administered directly to the patient without any need for repackaging.

In one embodiment, if the package contains a controlled substance, the package can be numbered sequentially for accountability purposes. Each package is configured such that contents can be delivered directly to the patient.

In one configuration, the blister pack layout is customized based on the specific nutrient compound and pharmaceutical drug combination provided to the system. This customization can include variations in compartment size, number of doses per pack, or additional labeling to provide patient instructions or identify the contents of each compartment.

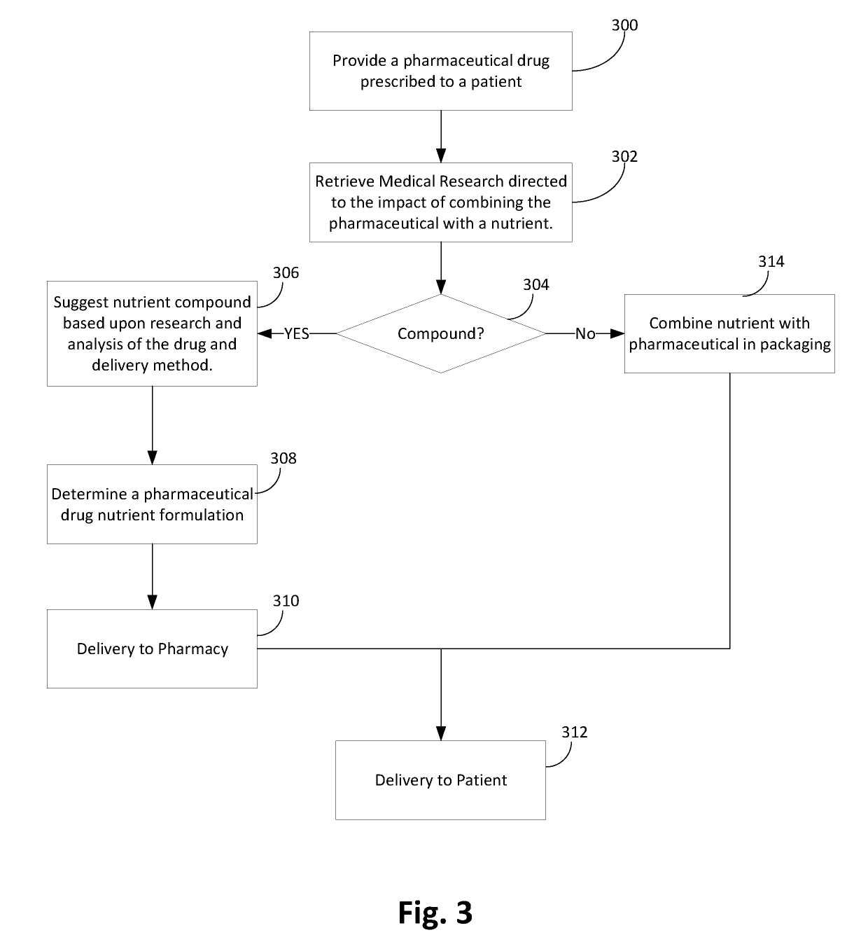

Referring to FIG. 3, a flowchart of a system for creating nutrient / pharmaceutical combination and compounds is shown. The process begins at a step 300, where a pharmaceutical drug prescribed to a patient is provided. The pharmaceutical drug at step 300 serves as the foundation for the nutrient compounded pharmaceutical and can be selected from chemotherapy drugs, opioid addiction treatment drugs, antidepressants, HIV drugs, cardiovascular disease drugs, diabetes drugs and others.

The process proceeds to a step 302, where medical research directed to the impact of combining the pharmaceutical drug with a nutrient is retrieved. The medical research at step 302 can include research, clinical findings, scientific findings and the like relevant to nutrient and pharmaceutical combinations, including the benefits of combining nutrients and pharmaceuticals to increase efficacy and decrease side effects. The medical research at step 302 can be retrieved from pharmaceutical databases, clinical trial repositories, peer-reviewed publications, and other sources.

Following the retrieval of research, the process moves to a step 304, which involves a decision point to determine whether to compound the pharmaceutical with nutrients. The decision at step 304 determines whether the nutrient and pharmaceutical drug are to be physically compounded into a single dosage form or provided separately in packaging.

If compounding is selected at step 304, the process proceeds to a step 306, where a nutrient compound is suggested based upon research and analysis of the drug and delivery method. The nutrient compound at step 306 can include vitamins to address deficiencies identified in the nutrient analysis of the research and database. The nutrient compound can include specific amino acids for inclusion when patient data analysis indicates a microbiome deficiency in bacteria that produce certain amino acids. The nutrient compound can include prebiotics or probiotics based on the results of patient data microbiome analyses. In one configuration, the nutrient compound includes Omega-3 fatty acids if the analyses suggest inflammation or a diet low in these essential fatty acids. In one embodiment, the nutrient compound includes antioxidants such as vitamin C or vitamin E if the metabolomics research and analysis indicate high levels of oxidative stress. Other nutrients can be used that are associated with conditions.

From step 306, the process continues to a step 308, where a pharmaceutical drug nutrient formulation is determined. The formulation at step 308 can include combining the pharmaceutical drug with the nutrient compound by incorporating the nutrient compound into a matrix of a tablet or capsule containing the pharmaceutical drug. In one configuration, the formulation at step 308 involves determining the appropriate dosages of both the pharmaceutical drug and the nutrient compound to complement the pharmaceutical treatment.

In some embodiments, empirical testing methods may be employed to determine the combination and dosage of the nutraceutical and pharmaceutical drug. Such empirical testing may be used to optimize dosage ratios and combinations to reduce side effects and increase efficacy. The empirical testing may include clinical studies, laboratory analyses, or other validation methods that assess the synergistic effects of the nutrient compound when combined with the pharmaceutical drug. In some cases, the empirical testing may identify optimal formulations by evaluating various dosage levels and nutrient combinations to determine configurations that provide enhanced therapeutic outcomes. The results of empirical testing may be incorporated into the system to refine formulation recommendations and improve the effectiveness of the nutrient-compounded pharmaceuticals.

The process then moves to a step 310, where the formulation is delivered to a pharmacy. The delivery at step 310 can include providing the formulation specifications to a compounding pharmacy for preparation of the nutrient compounded pharmaceutical.

If the decision at step 304 indicates that compounding is not selected, the process proceeds to a step 314, where the nutrient is combined with the pharmaceutical in packaging. At step 314, the system can create a co-packaged product where the pharmaceutical drug and nutrient compound are provided separately but intended to be taken together. The co-packaged product at step 314 can include blister packs, bags and other packaging with separate compartments that maintain the pharmaceutical drug and nutrient compound in separate but associated containers. The source of the pharmaceutical drug and nutrient combination or compound can be from the same vendor, different vendors and a combination.

Both paths from step 310 and step 314 converge at a step 312, where the nutrient compounded pharmaceutical or the combined package is delivered to the patient. The delivery at step 312 provides the patient with the nutrient and pharmaceutical combination in the selected format.

In one embodiment, the nutrient compounded pharmaceuticals are manufactured on a mass-scale. This approach leverages research results to create standardized nutrient-compounded formulations that can benefit a wide range of patients with similar conditions.

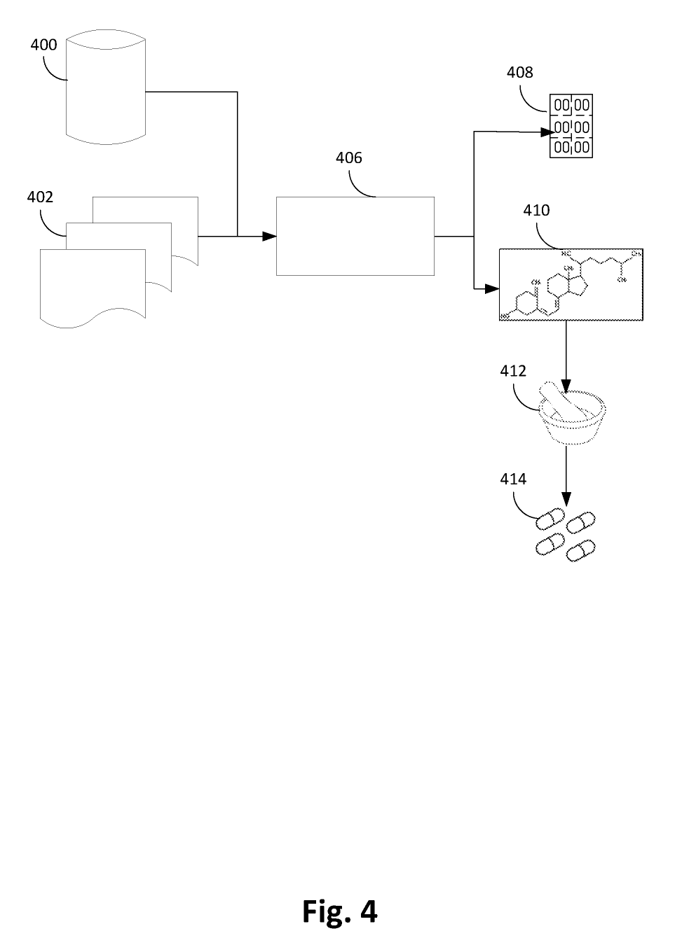

Referring to FIG. 4, a system diagram for creating nutrient compounded pharmaceuticals is shown. The system includes a pharmaceutical and nutrient database 400, which provides a comprehensive repository of information on drugs and nutrients. The pharmaceutical and nutrient database 400 can include a listing of pharmaceutical drugs with applications, doses, interactions, side effects, clinical trials, results, and other information. The pharmaceutical and nutrient database 400 can also include a listing of nutrients and nutraceuticals with applications, doses, interactions, side effects, clinical trials, results, impact on pharmaceutical drugs, synergistic impact, and other information. The pharmaceutical and nutrient database 400 can be a single database or can be several databases that can be accessed to receive pertinent information.

Medical research 402 provides research, clinical findings, and scientific findings relevant to nutrient and pharmaceutical combinations. The medical research 402 includes information regarding the benefits of combining nutrients and pharmaceuticals to increase efficacy and decrease side effects. In one example, the medical research 402 includes studies demonstrating that nutraceuticals can help patients who are at risk of conditions associated with certain pharmaceutical treatments.

A system 406 receives or retrieves information from the pharmaceutical and nutrient database 400 and the medical research 402. The system 406 processes and analyzes data from multiple sources to receive or determine a formulation or combination of the nutrient and pharmaceutical. The system 406 can rely on third-party sources for information to assemble the nutrient and pharmaceutical into a package 408 or to provide or create a formulation 410.

The package 408 represents the packaging of the nutrient and pharmaceutical combination for delivery to a patient. The formulation 410 stage determines the appropriate nutrient compounds based on received data. The formulation 410 involves selecting specific nutrients and determining dosages to complement the pharmaceutical treatment. In one embodiment, the formulation 410 includes combining the pharmaceutical drug with the nutrient compound by incorporating the nutrient compound into a matrix of a tablet or capsule containing the pharmaceutical drug.

Following formulation, compounding 412 includes the physical combination of the identified or selected compound materials. The compounding 412 stage physically combines the pharmaceutical drug and nutrient compound into a single dosage form.

Once compounded, a pharmaceutical and nutrient compound 414 represents the nutrient-compounded pharmaceutical product ready for administration to a patient. The pharmaceutical and nutrient compound 414 provides a final product that combines the pharmaceutical drug with specific dietary nutrients to enhance drug effectiveness and reduce side effects.

The system provides improvements to computer system functionality through automated retrieval and analysis of pharmaceutical and nutrient data from multiple databases. The system 406 receives or retrieves information from the pharmaceutical and nutrient database 400 and the medical research 402, processing and analyzing data that would otherwise require manual review by practitioners. The computerized system can employ machine learning algorithms that analyze research data, clinical outcomes, and patient feedback to identify patterns and correlations between specific nutrient compounds and pharmaceutical efficacy. The system utilizes natural language processing techniques to extract relevant information from unstructured research documents and integrate this information into the decision-making process. This automated analysis enables the system to process large volumes of research data and identify previously unknown synergistic relationships between nutrients and pharmaceutical drugs, as well as to detect potential adverse interactions that may not have been apparent through manual review. The iterative refinement capability allows the system to improve the accuracy and effectiveness of its recommendations as more data becomes available, resulting in increasingly optimized nutrient-compounded pharmaceutical suggestions over time.

In one embodiment, the system utilizes artificial intelligence (AI) or large language models (LLM) to analyze the pharmaceutical and nutrient data, identify patterns in research findings, and generate optimized formulation recommendations. The AI or LLM can process natural language from medical research publications and extract relevant information regarding nutrient-drug interactions to support the compounding determination process.

The system provides improvements to the pharmaceutical and healthcare industry by enabling the development of nutrient-compounded pharmaceuticals that can prevent adverse drug reactions, improve patient outcomes, reduce emergency room visits, and provide cost savings for patients and insurance providers. Certain pharmaceutical drugs reliably deplete specific nutrients in a large proportion of patients taking those medications, and the addition of the depleted nutrient to the pharmaceutical regimen can reduce side effects for a significant population of patients. For example, statins are known to deplete coenzyme Q10 in many patients, and proton pump inhibitors commonly reduce magnesium and vitamin B12 levels. By combining the pharmaceutical drug with the nutrient that is reliably depleted, the system can proactively address these deficiencies before they manifest as adverse effects. The system enables the creation of fixed combinations of pharmaceutical drugs and nutrients that reduce pill burden and improve patient adherence to treatment regimens. The system also provides for predictable synergy between pharmaceutical drugs and nutrient compounds, and enables non-specialized combinations that scale easily for widespread manufacturing and distribution.

In some cases, enhanced drug effectiveness through nutrient combinations may allow for smaller doses of the pharmaceutical drug to be prescribed while maintaining therapeutic efficacy. Reduced pharmaceutical dosing may decrease associated side effects, as many adverse reactions are dose-dependent. Lower required doses may also reduce medication costs for patients and insurance providers. In some aspects, patients experiencing fewer side effects may demonstrate improved compliance with treatment regimens, potentially leading to better therapeutic outcomes. The combination of reduced side effects and improved compliance may result in fewer doctor visits and emergency room visits, providing additional cost savings for patients and healthcare systems.

The system provides practical applications including generating formulation specifications for compounding pharmacies, generating packaging configurations for co-packaged products, and providing recommendations that can be delivered to pharmacies and patients. The formulation 410 stage determines the appropriate nutrient compounds based on received data, which may involve selecting specific nutrients and determining their optimal dosages to complement the pharmaceutical treatment. In one embodiment, the formulation 410 includes combining the pharmaceutical drug with the nutrient compound by incorporating the nutrient compound into a matrix of a tablet or capsule containing the pharmaceutical drug. The package 408 represents the packaging of the nutrient and pharmaceutical combination for delivery to a patient, which can include blister packs, bags, and other packaging with separate compartments that maintain the pharmaceutical drug and nutrient compound in separate but associated containers. The system can output compound formulations and packaging specifications that can be delivered to a pharmacy or manufacturing facility for preparation of the nutrient compounded pharmaceutical.

The system provides for particular treatment applications through specific drug-nutrient combinations that address known nutrient depletion patterns. In one embodiment, the system may generate a formulation combining statins with coenzyme Q10 to address statin-induced CoQ10 depletion, which may reduce muscle-related side effects commonly associated with statin therapy. In another embodiment, the system may generate a formulation combining proton pump inhibitors with magnesium and vitamin B12 to address PPI-induced deficiencies that may otherwise lead to neurological symptoms or cardiovascular complications. In one configuration, the system may generate a formulation combining metformin with vitamin B12 to address metformin-induced B12 depletion in diabetic patients. In some cases, the system may generate a formulation combining antidepressants with folate to enhance therapeutic efficacy, as folate has been shown to increase the effectiveness of antidepressant medications. In one embodiment, the system may generate a formulation combining chemotherapy drugs with omega-3 fatty acids to reduce inflammation and improve treatment response in cancer patients. These particular treatment applications represent specific therapeutic interventions that go beyond mere data analysis and provide tangible health benefits to patients.

In one embodiment, the system controls physical compounding equipment and packaging machinery to produce tangible pharmaceutical products. The system 406 may output control signals or specifications that direct the physical manufacturing process, including signals to compounding equipment that physically combines the pharmaceutical drug and nutrient compound into a single dosage form. The formulation 410 stage may generate machine-readable instructions that control tablet presses, encapsulation equipment, or other pharmaceutical manufacturing apparatus. The package 408 configuration may include specifications that direct blister pack forming equipment, filling machinery, and sealing apparatus to produce the co-packaged products. In one configuration, the system interfaces with automated dispensing equipment at compounding pharmacies to control the precise measurement and combination of pharmaceutical and nutrient components. The system may also generate control parameters for quality assurance equipment that verifies the proper composition of the nutrient-compounded pharmaceutical products. This integration with physical manufacturing equipment transforms the system's analytical outputs into tangible pharmaceutical products ready for patient administration.

The drug-nutrient combinations produced by the system may have markedly different characteristics from the individual components. In some cases, the combination of a pharmaceutical drug with a specific nutrient may result in different therapeutic properties than either component alone, such as enhanced bioavailability of the pharmaceutical drug when combined with certain nutrients that facilitate absorption. In one embodiment, the combination may exhibit a different side effect profile than the pharmaceutical drug alone, as the nutrient component may counteract or mitigate adverse effects that would otherwise occur. The combination may also demonstrate different pharmacokinetic properties, such as altered absorption rates, distribution patterns, or elimination half-lives compared to the pharmaceutical drug administered without the nutrient. In some configurations, the synergistic interaction between the pharmaceutical drug and the nutrient may produce therapeutic effects that exceed the additive effects of the individual components. These markedly different characteristics distinguish the nutrient-compounded pharmaceutical products from simple aggregations of their constituent parts and represent novel therapeutic compositions with distinct clinical utility.

The processes, methods, and functionality described herein are performed using computer readable instructions executed by a computerized system. The system 406 can include one or more processors that execute computer readable instructions stored in memory to perform the steps of retrieving pharmaceutical research, analyzing nutrient data, determining formulations, and generating packaging specifications. The computerized method can include receiving input data from the pharmaceutical and nutrient database 400 and the medical research 402, processing the data according to programmed algorithms, and outputting formulation recommendations or packaging configurations. The computer readable instructions can be stored on non-transitory computer readable media and can be executed to perform the nutrient analysis, pharmaceutical research retrieval, and compounding determination steps. In one configuration, the computerized system implements the decision logic at step 304 to determine whether to compound the pharmaceutical with nutrients or to combine the nutrient with the pharmaceutical in packaging. The computerized method can automatically retrieve and process medical research, suggest nutrient compounds, and generate formulation specifications for delivery to a pharmacy or manufacturing facility.

According to one embodiment, the processes, techniques and functionality described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the processes, techniques and functionality, or may include one or more general purpose hardware processors configured, adapted and programmed to perform the processes, techniques and functionality pursuant to program instructions, such as computer readable instructions, in firmware, memory, other storage, or a combination. Such special-purpose computing devices may also combine custom hard-wired logic, ASICs, or FPGAs with custom programming to accomplish the processes, techniques and functionality. The special-purpose computing devices may be desktop computer systems, portable computer systems, handheld devices, networking devices or any other device that incorporates hard-wired and/or program logic to implement the processes, techniques and functionality.

One or more different inventions may be described in the present application. Further, for one or more of the invention(s) described herein, numerous embodiments may be described in this patent application, and are presented for illustrative purposes only. The embodiments described are not intended to be limiting in any sense. One or more of the invention(s) may be widely applicable to numerous embodiments, as is readily apparent from the disclosure. These embodiments are described in sufficient detail to enable those skilled in the art to practice one or more of the invention(s), and it is to be understood that other embodiments may be utilized and that structural, logical, software, electrical and other changes may be made without departing from the scope of the one or more of the invention(s). Accordingly, those skilled in the art will recognize that the one or more of the invention(s) may be practiced with various modifications and alterations. Particular features of one or more of the invention(s) may be described with reference to one or more particular embodiments or figures that form a part of the present disclosure, and in which are shown, by way of illustration, specific embodiments of one or more of the invention(s). It should be understood, however, that such features are not limited to usage in the one or more particular embodiments or figures with reference to which they are described. The present disclosure is neither a literal description of all embodiments of one or more of the invention(s) nor a listing of features of one or more of the invention(s) that must be present in all embodiments.

Headings of sections provided in this patent application and the title of this patent application are for convenience only, and are not to be taken as limiting the disclosure in any way.

It is understood that the above descriptions and illustrations are intended to be illustrative and not restrictive. It is to be understood that changes and variations may be made without departing from the spirit or scope of the following claims. Other embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventor did not consider such subject matter to be part of the disclosed inventive subject matter.

What is claimed is:

1. A computer system for developing and providing a drug-nutrient combination, comprising:
a computerized system having a processor and a memory storing instructions that, when executed by the processor, cause the system to:
retrieve pharmaceutical research from one or more databases, the pharmaceutical research including information regarding a pharmaceutical drug, drug mechanisms of action, potential side effects, and dosing;
retrieve medical research directed to an impact of combining the pharmaceutical drug with a nutrient;
analyze the pharmaceutical research and the medical research to identify a nutrient having a synergistic impact on the pharmaceutical drug;
determine a drug-nutrient combination based on the pharmaceutical research and the medical research, the drug-nutrient combination including the pharmaceutical drug and the nutrient; and
generate a recommendation for the drug-nutrient combination.

2. The computer system of claim 1, including retrieving a nutrient analysis data including information regarding nutritional deficiencies associated with the pharmaceutical drug.

3. The computer system of claim 1, wherein the computer system is adapted to retrieve patient analysis data including disease-related patient research to identify appropriate nutrients for a patient population.

4. The computer system of claim 1, wherein the nutrient is selected from the group consisting of: vitamins, minerals, amino acids, omega-3 fatty acids, prebiotics, probiotics, antioxidants and any combination thereof.

5. The computer system of claim 1, wherein the pharmaceutical drug is selected from the group consisting of: chemotherapy drugs, opioid addiction treatment drugs, antidepressants, HIV drugs, cardiovascular disease drugs, diabetes drugs and any combination thereof.

6. The computer system of claim 1, wherein the computer system is adapted to generate a packaging specification for co-packaging the pharmaceutical drug and the nutrient in a compartment of a package.

7. The computer system of claim 1, wherein the drug-nutrient combination enables a reduced dose of the pharmaceutical drug to be prescribed while maintaining therapeutic efficacy.

8. A computer system for identifying a compound, comprising:
a computer system having a processor and a set of computer readable instructions that, when executed by the processor, cause the system to:
access a pharmaceutical and nutrient database including listings of pharmaceutical drugs with applications, doses, interactions, and side effects, and listings of nutrients with applications, doses, and synergistic effects with pharmaceutical drugs;
receive an indication of a pharmaceutical drug;
retrieve, from the pharmaceutical and nutrient database, information regarding the pharmaceutical drug and one or more nutrients;
evaluate compound properties including an interaction between the pharmaceutical drug and a nutrient;
determine suitability of a compound including the pharmaceutical drug and a selected nutrient based on the compound properties; and
output a compound formulation including the pharmaceutical drug and the selected nutrient.

9. The computer system of claim 8, wherein the compound formulation includes a dose of the pharmaceutical drug and an amount of the selected nutrient configured to provide a synergistic effect.

10. The computer system of claim 8, wherein the computer system id adapted to retrieve medical research including clinical findings relevant to combining the pharmaceutical drug with the one or more nutrients.

11. The computer system of claim 8, wherein the compound formulation specifies incorporating the selected nutrient into a matrix of a tablet or capsule containing the pharmaceutical drug.

12. The computer system of claim 8, wherein the compound formulation enables a reduced dose of the pharmaceutical drug while maintaining therapeutic efficacy.

13. A computerized system for providing a drug-nutrient combination, comprising:
a computer system having a processor and a set of computer readable instructions that, when executed by the processor, cause the system to:
receive information from a pharmaceutical and nutrient database and medical research;
process and analyze the information to determine a formulation or combination of a nutrient and a pharmaceutical drug;
determine whether to compound the pharmaceutical drug with the nutrient into a single dosage form or to combine the nutrient with the pharmaceutical drug in packaging;
generate a formulation for delivery to a pharmacy, the formulation including the pharmaceutical drug combined with the nutrient, based on a determination to compound; and
generate a packaging configuration including the pharmaceutical drug and the nutrient in separate compartments, based on a determination to combine in packaging, .

14. The computerized system of claim 13, wherein the packaging configuration includes a blister pack having a packet section divided into multiple compartments, each compartment containing a nutraceutical and a pharmaceutical drug.

15. The computerized system of claim 13, wherein the computer system is adapted to utilize natural language processing techniques to extract information from research documents and integrate the information into a decision-making process for determining the formulation or combination.

16. The computerized system of claim 13, wherein the formulation or combination enables a reduced dose of the pharmaceutical drug while maintaining therapeutic efficacy.

17. A method for developing and providing a drug-nutrient combination, comprising:
retrieving, by a processor, pharmaceutical research from one or more databases, the pharmaceutical research including information regarding a pharmaceutical drug, drug mechanisms of action, potential side effects, and dosing;
retrieving, by the processor, medical research directed to an impact of combining the pharmaceutical drug with a nutrient;
analyzing, by the processor, the pharmaceutical research and the medical research to identify a nutrient having a synergistic impact on the pharmaceutical drug;
determining, by the processor, a drug-nutrient combination based on the pharmaceutical research and the medical research, the drug-nutrient combination including the pharmaceutical drug and the nutrient; and
generating, by the processor, a recommendation for the drug-nutrient combination.

18. The method of claim 17, further comprising retrieving nutrient analysis data including information regarding nutritional deficiencies associated with the pharmaceutical drug; and selecting the nutrient based on the nutrient analysis data to address the nutritional deficiencies.

19. The method of claim 17, wherein the drug-nutrient combination enables a reduced dose of the pharmaceutical drug to be prescribed while maintaining therapeutic efficacy.

20. The method of claim 17, including creating the drug-nutrient combination.

21. The method of claim 20, including distributing the drug-nutrient combination to a health care provider.

22. The method of claim 20, including distributing the drug-nutrient combination to a patient.

23. A method for providing a drug-nutrient combination, comprising:
analyzing a pharmaceutical research from one or more databases, the pharmaceutical research including information regarding a pharmaceutical drug, drug mechanisms of action, potential side effects, and dosing;
analyzing a medical research directed to an impact of combining the pharmaceutical drug with a nutrient;
determining a drug-nutrient combination based on the pharmaceutical research and the medical research, the drug-nutrient combination including the pharmaceutical drug and the nutrient wherein the nutrient includes a synergistic impact on the pharmaceutical drug; and,
generating a recommendation for the drug-nutrient combination.

24. The method of claim 23, including creating the drug-nutrient combination.

25. The method of claim 24, including distributing the drug-nutrient combination to a health care provider.

26. The method of claim 24, including distributing the drug-nutrient combination to a patient.
